(19) United States
(12) Patent Application Publication
(10) Pub. No.: US 2011/0160305 A1
(43) Pub. Date: Jun. 30, 2011

(54) Oral Delivery of Therapeutic Doses of Glutathione

(76) Inventor: Marty Jones, Lakewood, CO (US)

(21) Appl. No.: 11/743,138

(22) Filed: May 1, 2007

(51) Int. Cl. A61K 31/225; A61K 31/197; A61P 25/28; A61P 1/16; A61P 25/16; A61P 31/18; A61P 35/00; A61P 9/00; A61P 3/10

(52) U.S. Cl. 514/547; 514/562

(57) ABSTRACT

Nutritional supplements such as acetyl glutathione with malodor treated to remove the malodor associated with it. Acetyl glutathione, glutathione, esters of glutathione, derivatives of glutathione, and precursors of glutathione and other thiol compounds that may have a strong malodor based on their sulfur content. These compounds are treated with tartaric acid and other products to remove this malodor.

### FIELD OF THE INVENTION

This invention relates to the field of oral delivery of glutathione and other nutritional supplements.

### BACKGROUND OF THE INVENTION

Glutathione (gamma-glutamyl-cysteinyl-glycine) has been described as an essential antioxidant, antitoxin and protector of DNA and cellular health. There have been many peer reviewed scientific papers published that discuss the relationship between reduced glutathione levels and the progression of many chronic diseases. Deficiencies of glutathione contributes to hemolysis (break down of red blood cells), oxidative stress which plays a key role in aging, and the progression of many diseases, including Alzheimer's disease, Parkinson's disease, liver disease, cystic fibrosis, sickle cell anemia, HIV, AIDS, cancer, heart attach, stroke, diabetes and many others. It is also believed to be of use in treating a variety of issues such as drug induced hepatoxicity, illnesses such as influenza, diseases such as herpes and HIV, as well as physical problems including vascular, ocular and auditory problems. Glutathione is vital to biological systems and regulates many signaling pathways via its reducing capabilities.

Glutathione is a very important tripeptide thiol (protein building blocks containing the functional group composed of a sulfur atom and a hydrogen atom) that is well known for its antioxidant properties as well as its importance as a catalyst, reductant and a reactant. It is often referred to as the master antioxidant or the primary antioxidant. Glutathione is built from the amino acids glycine, cysteine and glutamate. The synthesis of these three amino acids is catalyzed sequentially by two cytosolic enzymes, gammaglutamycystein synthetase and GSH synthetase. It exists in a reduced form referred to as GSH and an oxidized form, glutathione disulfide referred to as GSSG. Glutathione is abundant in cytoplasm, nuclei and in mitochondria.

Reduced glutathione (GSH) is produced in humans from oxidized glutathione (GSSG) primarily by the liver. Oxidized glutathione is constitutively active and may be induced by oxidative stress leading to a deficiency of GSH. A considerable amount of glutathione may become protein bound during severe oxidative stress. Also, the presence of many toxins may inhibit the production of GSH. The replenishment of GSH by the human body is challenging. GSH has a short half-life in blood plasma and is not taken up by the cells directly. While the human body is capable of synthesizing GSH, often it is desirable to increase the amount of GSH available for reducing cellular oxidative stresses, preventing cellular oxidation, repair cellular damage and for many other important functions.

Glutathione is usually administered intravenously or through alveolar inhalation. These protocols are expensive, uncomfortable and largely impractical which cause their use only in extreme situations. An oral dosage of glutathione is desirable since it is easy to administer, likely to be less expensive and could be administered personally.

A problem with oral delivery of glutathione is that glutathione is difficult to ingest orally. It is digested by the stomach since it is protein based. Also, glutathione is unstable in alkaline or oxidative environments and degraded in the stomach by the desulfurases and peptidases. Oral administration of glutathione is not considered effective because of hydrolysis of glutathione by intestinal and hepatic gamma-glutamyltransferase. The amount of oral doses of glutathione that would be needed to be effective due to its poor gastrointestinal absorption could lead to harmful side effects.

One approach to overcoming the poor results from oral delivery of glutathione has been the use of oral delivery of glutathione precursors. The precursor that is commonly used is cysteine. Cysteine is not considered an essential amino acid so it is usually in less abundance in the diet of most humans and is considered the rate limiting factor in the in vivo production of GSH. Cysteine, as a free amino acid, is potentially toxic and is spontaneously catabolized or destroyed in the gastrointestinal tract and blood plasma. N-acetyl cysteine is also used in place of cysteine is used for this reason. Cysteine and N-acetyl cysteine also compete with existing glutathione for resources in certain reducing pathways.

However, when it is present as a cysteine-cysteine dipeptide, called cystine, it is more stable than cysteine and is reduced to the two cysteine molecules upon cell entry. There are concerns with cystine including the ability of the body to absorb it, bladder or kidney stones, the possibility of cystinosis and the production of homocysteine. Also, the bonds of cystine may be split by heat, low pH and mechanical stress and lead to toxicity and destruction.

These obstacles may be overcome by a glutathione derivative S-acetylglutathione (S-GSH), which is more stable in plasma and taken up directly by cells with subsequent conversion to GSH. S-GSH is formed by acetylaing glutathione such as described in U.S. Pat. No. 5,382,679. An example of forming S-GSH is by reacting glutathione or esters or precursors thereof with reagents such as acyl halides or acid anhydrides. Other methods of forming S-GSH or other acetyl derivatives of glutathione or its esters are known as well. S-GSH is much more stable, able to bypass digestion in the stomach and does not negatively affect the production of GSH in the cells. It is thus able to be transported directly into the cells for conversion to GSH.

One significant problem with the oral use of certain supplements or compounds is malodor. The odor of many thiols, sulfur based compounds and other compounds is often strong and repulsive, particularly for those of low molecular weight. For example, some sulfur based compounds are responsible for the intolerable, persistent odor produced by feces, rotting flesh and the spraying of skunks. These malodors arise primarily from volatile thiol and hydrogen sulfide degradation products found within the compositions. Unfortunately, sulfur containing compositions may be, or may become, malodorous over time yielding a characteristic “rotten egg odor.” As a result of the unpleasant odor, patients are sometimes reluctant to apply such compositions directed by their physician or pharmacist. Such poor patient compliance can seriously diminish the effectiveness of a treatment regimen.

These odor problems exist not only in nutritional supplements containing free sulfhydryls but in other malodorous nutritional compounds as well as topical compounds containing free sulfhydryl and other malodorous compounds. There are certain forms of glutathione that have this issue with malodor which limits their effective use in oral dosages. For example, acetylated glutathione with the sulfhydryl exposed has a particularly offensive odor.

Thus a problem exists in the malodorous nutritional and topical compounds and particularly with the oral administration of glutathione and its derivatives, esters and precursors that may have malodor issues.

### SUMMARY OF THE INVENTION

The present invention solves these and other problems by providing a glutathione product that has been treated to remove the malodor associated with it. The glutathione product includes GSH, SGSH, partially acetylated glutathione, glutathione esters, derivatives of glutathione and precursors of glutathione including cystine that may have malodor. The treated compound has the malodor minimized or even eliminated. This greatly increases the ability of individuals to tolerate the compound so that the compliance with the dosage is improved.

One preferred embodiment of the present invention utilizes acetyl glutathione. Acetyl glutathione is much more stable that GSH, less likely to react within the stomach and less likely to be absorbed by the stomach, more likely to be transported directly to the cells and able to be converted to GSH at the cellular level. However, typical acetyl glutathione is an extremely thick, sticky mass and unmanageable so to be difficult to work with and difficult to take orally. The acetyl glutathione of the preferred embodiment of the present invention has the free sulfhydryl exposed. This embodiment of acetyl glutathione has either glycine or glutamate acetylated. The cysteine moiety is not acetylated. This embodiment is for purposes of this application referred to as N-acetyl glutathione. This provides a liquid form that is less viscous and easier to work with and to take orally. Unfortunately, this form of acetyl glutathione is extremely malodorous to the point that it is nearly impossible to tolerate.

The N-acetyl glutathione, in this preferred embodiment of the present invention, is compounded with tartaric acid to remove the extremely strong and noxious malodor associated with it. The final product is a liquid without little or no smell and thus able to be easily tolerated by the individual.

While the above embodiment describes the compounding of an embodiment of acetylated glutathione where the cysteine moiety is not acetylated with tartaric acid, other forms of glutathione, precursors of glutathione such as cysteine and cystine and esters of glutathione that may have malodor may also be used in a preferred embodiment of the present invention. These compounds are combined with tartaric acid to remove the malodor associated with them.

While not all thiol compounds may have malodor, many do. These thiol compounds with malodor may be combined with tartaric acid in other preferred embodiments of the present invention. The tartaric acid minimizes or eliminates the strong malodor that is associated with these compounds to increase their tolerability.

The present invention, in a preferred embodiment, encompasses other nutritional supplements that have strong malodor. These supplements are combined with tartaric acid and its equivalents to remove the malodor.

Orally delivered medications with strong malodors may also be covered under the present invention. These medications may be combined with tartaric acid to remove the malodor.

Another preferred embodiment of the present invention includes topically applied compounds containing thiol compounds that may have malodor and other ingredients with malodor or that develop malodor upon exposure. These compounds are combined with tartaric acid to remove the malodor or the development of malodor.

These and other features of the present invention are found in the ensuing description of preferred embodiments and from the claims.

### DETAILED DESCRIPTION OF PREFERRED EMBODIMENTS

A preferred embodiment of the present invention is discussed herein. It is to be expressly understood that the descriptive embodiments are provided herein for explanatory purposes only and are not meant to unduly limit the claimed inventions. The exemplary embodiments describe the present invention in terms of a nutritional supplement containing glutathione and glutathione derivatives and precursors for oral delivery. It is to be expressly understood that other supplements may be used under the present invention as well as topical application compounds.

As used herein, a thiol is a compound that contains the functional group composed of a sulfur atom and a hydrogen atom (—SH). This functional group is referred to either as a thiol group or a sulfhydryl group. Some specific examples of thiols, dithiols, and similar malodor compounds include, but are not limited to, glyceryl monomercaptan, thioglycolic acid, cysteine, cysteine esters, N-acetyl-cysteine, N-acetyl-cysteine esters; N,S-diacetyl-cysteine esters, glutathione, glutathione esters, N-acetyl-glutathione, N-acetyl-glutathione esters, methionine, ammonium thioglycolate, calcium thioglycolate, zinc thioglycolate, potassiumm thioglycolate, monoethanolammonium thioglycolate, mercaptopurine, lipoic acid, and 6,8-dimercaptooctanoic acid (dihydrolipoic acid).

Not all of these thiol compounds have issues with odors, but many do, particularly those compounds that have a free sulfhydryl. The malodors produced by these compounds range in degrees from rotten eggs, decomposed fish, dead animals, to substances ejected by a skunk.

A preferred embodiment of the present invention compounds thiol compounds having malodor with tartaric acid (2,3-dihydroxybutanedioc acid). Tartaric acid is a white crystalline organic acid. It occurs naturally in many plants, particularly grapes, bananas, and tamarinds, and is one of the main acids found in wine. It is added to other foods to give a sour taste, and is used as an antioxidant. Salts of tartaric acid are known as tartrates. It is a dihydroxy derivative of dicarboxylic acid.

It is to be expressly understood that other compounds including other dicarboxylic acid that have been deemed as Generally Recognized As Safe (GRAS) may be used in the present invention as well to remove the malodor of nutritional supplements, topically applied compounds and other compounds that may have malodor associated therewith. Examples of these dicarboxylic acid includes without limitation adipic, aldaric, aspartic, fumaric, ketogultaric, malic, malonic, and others.

One example of a preferred embodiment of the present invention is discussed here. Glutathione (GSH) is acetylated to form acetyl glutathione, N-acetyl glutathione. This can be done by several different methods that are not of particular concern to this example. The typical resulting N-acetyl glutathione is a thick sticky mass that is difficult to work with and handle. The preferred embodiment of the present invention solves this problem by not acetylating the cysteine moiety. Either the glycine or glutamate is acetylated, but not cysteine. This provides a liquid that is easy to formulate into a liquid dose. Unfortunately, the resulting liquid, referred to herein as N-acetyl glutathione, has a distinct and unpleasant odor that renders it virtually unusable.

In the preferred embodiment of the present invention, N-acetyl glutathione is compounded with tartaric acid ((2,3-dihydroxybutanedioc acid). In this preferred embodiment 42 milliliters of N-acetyl glutathione is compounded with 15 grams of tartaric acid. Other ranges of the N-acetyl glutathione as well as tartaric acid may be used as well. Additional compounds may be added as well, such as surfactants, stabilizers, binders and others may be added as well to create a usable product.

The resulting product contains an effective oral dosage of N-acetyl glutathione that is palatable for human use. The malodor has been greatly reduced if not eliminated. The acetyl glutathione is essentially tasteless so that compliance by the user with oral delivery of N-acetyl glutathione is increased. Oral delivery of the N-acetyl glutathione increases the availability of dietary glutathione for most individuals as compared to the intravenous and inhalation methods previously used. Also the use of N-acetyl glutathione with tartaric acid provides a highly efficient delivery of N-acetyl glutathione at the cellular level where it can be easily converted to reduced glutathione.

The resulting product is delivered much more efficiently and comfortably than previous GSH products. This increases the delivery of these GSH products to the cellular level to increase the production and/or use of GSH at the cellular level to reduce oxidative stress, to increase protection of the cells, to treat the vast number of diseases and disorders that GSH may be effective with treating and the many other beneficial results from GSH.

The resulting product of N-acetyl glutathione with tartaric acid is also easy to formulate into a powder form as well as liquid. This enables it to be easily produced in tablet or capsules for ease of oral delivery. Another important feature is the ability to control the dosage amounts by providing the product in tablets or capsules. Also, additional ingredients can be added to provide additional benefits and to increase the storage life.

The resulting product of N-acetyl glutathione with tartaric acid may also be administered in many other forms as well. Examples of these types of administration without limitation include sublingual, inhaled, topical, rectally, vaginally, injection and any other type of delivery.

Tartaric acid may also be combined with other forms of glutathione including esters of glutathione, derivatives of glutathione and precursors of glutathione such as cystine that may have issues with malodor. The tartaric acid minimizes the malodor associated with these products thus providing a greater range of glutathione delivery methods. Thus the use of GSH in treating the limitless range of disorders can be greatly increased.

The use of tartaric acid may be used with other thiol compounds that may have malodor such as but not limited to glyceryl monomercaptan, thioglycolic acid, cysteine, cysteine esters, N-acetyl-cysteine, N-acetyl-cysteine esters; N,S-diacetyl-cysteine esters, methionine, ammonium thioglycolate, calcium thioglycolate, zinc thioglycolate, potassiumm thioglycolate, monoethanolammonium thioglycolate, mercaptopurine, lipoic acid, and 6,8-dimercaptooctanoic acid (dihydrolipoic acid). These may be used with tartaric acid to minimize the malodor associated with these compounds. Other thiol compounds with malodor may be combined with tartaric acid in other preferred embodiments of the present invention. The tartaric acid minimizes or eliminates the strong malodor that is associated with these compounds to increase their tolerability.

The present invention, in a preferred embodiment, encompasses other nutritional supplements that have strong malodor. These supplements are combined with tartaric acid and its equivalents to remove the malodor.

Orally delivered medications with strong malodors may also be covered under the present invention. These medications may be combined with tartaric acid to remove the malodor.

Tartaric acid may also be used with topically applied compounds. Glutathione and other thiol compounds have been found to have efficacy in treating skin disorders and for cosmetic applications. However, many thiol compounds have malodor that affects compliance with their use. Also, many thiol compounds that do not normally have a malodor combine with the proteins in the skin to develop a severe sulfur odor. The use of tartaric acid with these compounds minimize or even eliminate this odor.

These and other compounds may be combined with tartaric acid and its equivalents to create a non-offensive smelling product. These and other embodiments of the present invention are considered to be within the scope of the presently claimed invention.

What is claimed is:

1. A nutritional supplement for oral delivery, said supplement comprising:
a thiol compound having malodor; and
tartaric acid.

2. The nutritional supplement of claim 1, said thiol compound includes:
glutathione compounds with malodor.

3. The nutritional supplement of claim 1, said thiol compound includes:
reduced glutathione with malodor.

4. The nutritional supplement of claim 1, said thiol compound includes:
acetyl glutathione.

5. The nutritional supplement of claim 1, said thiol compound includes:
acetyl glutathione with a free sulfhydryl.

6. The nutritional supplement of claim 1, said thiol compound includes:
acetyl glutathione with cysteine not acetylated.

7. The nutritional supplement of claim 1, said thiol compound includes:
glutathione derivatives with malodor.

8. The nutritional supplement of claim 1, said thiol compound includes:
glutathione precursors with malodor.

9. The nutritional supplement of claim 1, said thiol compound includes:
esters of glutathione with malodor.

10. The nutritional supplement of claim 1 wherein said supplement includes:
said nutritional supplement formed in tablet form.

11. The nutritional supplement of claim 1 wherein said nutritional supplement includes:
said nutritional supplement is provided for delivery by one of the following delivery systems: oral, sublingual, inhaled, topical, rectally, vaginally, and injection.

12. A nutritional supplement, said nutritional supplement comprises:
a glutathione product having malodor; and
said glutathione product compounded with tartaric acid.

13. The nutritional supplement of claim 12 wherein said glutathione product having malodor includes:
acetylated glutathione with a free sulfhydryl.

14. The nutritional supplement of claim 12 wherein said glutathione product having malodor includes:
acetylated glutathione with the cysteine moiety not acetylated.

15. The nutritional supplement of claim 12 wherein said nutritional supplement includes:
said nutritional supplement is provided in tablet form for oral delivery.

16. The nutritional supplement of claim 12 wherein said nutritional supplement includes:
said nutritional supplement is provided for delivery by one of the following delivery systems: oral, sublingual, inhaled, topical, rectally, vaginally, and injection.

17. A nutritional supplement, said nutritional supplement comprises:
acetylated glutathione with a free sulfhydryl compounded with tartaric acid.

18. The nutritional supplement of claim 12 wherein said acetylated glutathione with a free sulfhydryl includes:
the cysteine moiety not acetylated.

19. The nutritional supplement of claim 12 wherein said nutritional supplement includes:
said nutritional supplement is provided in tablet form for oral delivery.
